(19) United States
(12) Patent Application Publication
(10) Pub. No.: US 2010/0119560 A1
(43) Pub. Date: May 13, 2010

(54) NANO-EMULSION, THE USE THEREOF, AND PREPARATION METHOD THEREOF

(76) Inventors: Young Dae Kim, Seoul (KR); Keun Ja Park, Seoul (KR); Jung Soo Kim, Seoul (KR); Ji Soo Kim, Seoul (KR)

Correspondence Address: KNOBBE MARTENS OLSON & BEAR LLP, 2040 MAIN STREET, FOURTEENTH FLOOR, IRVINE, CA, 92614, US

(21) Appl. No.: 11/682,279

(22) Filed: Mar. 5, 2007

Related U.S. Application Data
(63) Continuation-in-part of application No. PCT/KR05/02929, filed on Sep. 3, 2005.

(30) Foreign Application Priority Data
Sep. 4, 2004 (KR) 10-2004-0070665

(51) Int. Cl. A61K 8/02; A61K 31/355; A61Q 19/00

(52) U.S. Cl. 424/401; 514/458

(57) ABSTRACT

Embodiments of the present invention relate to nanoemulsions, the use thereof and preparation method thereof wherein the nanoemulsion contains a crosspolymer comprising at least one of a polyacrylic acid chain and a derivative of the polyacrylic acid chain; and polyoxypropylene-polyoxyethylene compound. According to one embodiment, the nanoemulsion forms a droplet with a diameter in the range of about 43 nm to about 96 nm.

### CROSS-REFERENCE TO RELATED APPLICATIONS

This application is a continuation-in-part application under 35 U.S.C. §365(c) of International Application No. PCT/KR2005/002929, filed Sep. 3, 2005, designating the United States. International Application No. PCT/KR2005/002929 was published in English as WO 2006/028339 A1 on Mar. 16, 2006. This application further claims for the benefit of the earlier filing dates under 35 U.S.C. §365(b) of Korean Patent Application No. 10-2004-0070665 filed Sep. 4, 2004. This application incorporates herein by reference the International Application No. PCT/KR2005/002929 including WO 2006/028339 A1 and the Korean Patent Application No. 10-2004-0070665 in their entirety.

### BACKGROUND

1. Field

The present disclosure relates to nanoemulsions, their use, and their preparation methods. More precisely, the invention relates to cosmetic compositions and their preparation methods of oil-in-water (o/w) nanoemulsions.

2. Description of the Related Technology

An emulsion is a non-homogeneous system in which one liquid is dispersed closely in another as a droplet. The mean droplet diameter of emulsions generally exceeds 0.1 μm but droplet diameters of emulsions in practical use are in the range of 0.5 to 100 μm. Emulsions have some disadvantages such as thermodynamic instability and poor absorption ability into the skin due to their large droplet size.

Smaller droplet sizes of emulsions not only suppress the coalescence or coagulation of emulsion droplets but also suppress the precipitation of emulsions and while further helping to deliver the active agents into the skin.

But in the case of nanoemulsions, thermodynamic stability is highly improved and the skin absorption ability of the active agents is also improved since the droplet size of nanoemulsions is much smaller than that of emulsions and the surface area of nanoemulsions is much larger than that of emulsions. The droplet size of nanoemulsions is defined differently depending on the author. In general the droplet size of nanoemulsions is defined as between 20 nm and 500 nm [Flockhart, I. R. etc, Nanoemulsions derived from lanolin show promising drug delivery properties, J. Pharm. Pharmacol. 50 (Supplement) 1998, 141].

There are generally two processes for the production of nanoemulsions. The first and most common procedure is to use a high pressure homogenizer at high pressures along with surfactants and co-surfactants. The second procedure is to use the phase inversion temperature (PIT) principle.

But in the case of the procedure using a high pressure homogenizer there are some problems such as poor productivity and component deterioration due to difficult mass production and generation of much heat, respectively, since nanoemulsions should be prepared by contacting an oil phase with a water phase instantly through a narrow valve slit under a high pressure, for example, under 500-1,600 atmospheric pressure.

And since low viscosity emulsions can only be manufactured by this procedure, high viscosity nanoemulsion creams cannot be prepared because of the poor miscibility and aggregation of the phases. Therefore, by this method generally only oil in water (o/w) liquid nanoemulsions of less than 20% oil phase can be prepared, while cream nanoemulsions of high viscosity or hardness with a mean droplet diameter lower than 200 nm cannot be prepared.

And in the case of the latter method of using a phase-inversion principle it is also difficult to control the PIT and the rapid cooling process during preparation. Nanoemulsions prepared using the PIT principle also have an instability problem of phase inversion near PIT since emulsions prepared by this method inverse at PIT easily.

Even though there are many problems as described above, nanoemulsions of low viscosity with a high amount of phospholipids as emulsifiers are mainly manufactured by using a high pressure homogenizer. And since the fluidity of droplets for instant contacting, mixing and the adsorption of surfactants at interfaces are important in the procedure of using a high pressure homogenizer, the use of polymeric thickeners like carbomers which decrease the fluidity of droplets and may hinder the adsorption of surfactants at interfaces was avoided.

The production of very small droplets in emulsions requires a large amount of energy and reduction of surface tension. Particularly, since there were not any good methods utilizing energy efficiently the emulsification method of using the inefficient high pressure homogenizer was mainly employed. But in this method a safety problem was triggered by the use of large amounts of surfactants. These problems were the main causes limiting the practical application of nanoemulsions in cosmetics, drugs, etc.

The foregoing discussion is simply to provide background information of the invention and does not constitute an admission of prior art.

### SUMMARY

According to an embodiment of the invention, a nanoemulsion may comprise a crosspolymer comprising at least one of a polyacrylic acid chain and a derivative of the polyacrylic acid chain; and a compound represented by Formula 1:

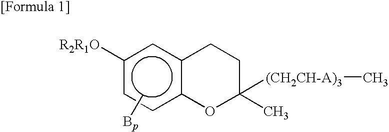

wherein R1 is —(O—CH2-CH2)m-, m is an integer from 0 to 300, wherein R2 is H—(O—CH(CH3)-CH₂)ₙ—, n is an integer from 1 to 250, wherein A is —CH₂—CH(CH₃)— or —C(CH3)=CH—, and wherein B is —CH3, and p is 1, 2 or 3.

According to an embodiment of the invention, the crosspolymer may comprise at least one selected from the group consisting of: a crosspolymer of a polyacrylic acid chain of Formula 2 crosslinked with an allyl ether of a polylol; a crosspolymer of a polyacrylic acid chain of Formula 2 crosslinked with an allyl ether of a propylene, and a C₆-C₄₀ alkyl acrylate polymer:

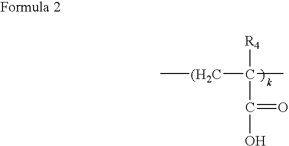

wherein R₄ is H or CH₃, and k is an integer from about 10 to about 100,000.

According to an embodiment of the invention, the polyol may be a pentaerythritol or a sucrose.

According to an embodiment of the invention, the compound of Formula 1 may comprise a natural vitamin E or a synthetic vitamin E.

According to an embodiment of the invention, the compound of Formula 1 may be present in an amount from about 0.5 to about 60 wt. % with reference to the total weight of the nanoemulsion.

According to an embodiment of the invention, the compound of Formula 1 may be present in an amount from about 1 to about 30 wt. % with reference to the total weight of the nanoemulsion.

According to an embodiment of the invention, the crosspolymer may be present in an amount from about 0.01 to about 40% with reference to the total weight of the nanoemulsion.

According to an embodiment of the invention, the crosspolymer may be present in an amount from about 0.02 to about 20% with reference to the total weight of the nanoemulsion.

According to an embodiment of the invention, the nanoemulsion may comprise a droplet with a diameter in the range of about 43 to about 96 nm.

According to an embodiment of the invention, a cosmetic composition may comprise the aforementioned nanoemulsion.

According to an embodiment of the invention, the aforementioned cosmetic composition may be in the form of a cream or a lotion for applying to skin.

According to an embodiment of the invention, the nanoemulsion may comprise at least one hydrophobic material selected from the group consisting of: paraffin oil, squalane, caprylic triglyceride, capric triglyceride, cetyloctanoate, octyldodecanol, isopropyl palmitate, jojoba oil, olive oil, safflower oil, evening primrose oil, Chinese pepper oil, sesame oil, shark oil, and oil soluble vitamins.

According to an embodiment of the invention, the nanoemulsion may further comprise at least one hydrophilic material selected from the group consisting of propylene glycol, butylene glycol, glycerine, polyethylene glycol, hyaluronic acid, condroitin sulfate, glucosamine, vitamin C and panthenol.

According to an embodiment of the invention, a method of treating skin may comprise providing the aforementioned nanoemulsion; and applying the nanoemulsion to skin.

According to another embodiment of the invention, a method of preparing the nanoemulsion may comprise providing a hydrophobic material, a hydrophilic material, the crosspolymer and the compound represented by Formula 1; and mixing the hydrophobic material, the hydrophilic material, the crosspolymer and the compound represented by Formula 1.

According to another embodiment of the invention, the weight ratio (φ) of the hydrophobic material to the total weight of the hydrophobic and hydrophilic materials may be from about 0.4 to about 0.75.

According to another embodiment of the invention, the high speed mixing may occur at 1 atmospheric pressure, and is not a high-pressure homogenizer.

According to another embodiment of the invention, a nanoemulsion comprises a compound represented by Formula 1:

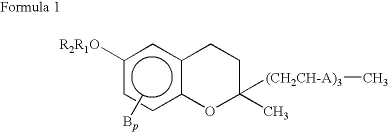

wherein R₁ is —(O—CH₂—CH₂)ₘ—, m is an integer from 0 to 300, wherein R₂ is H—(O—CH(CH₃)—CH₂)ₙ—, n is an integer from 1 to 250, wherein A is —CH₂—CH(CH₃)— or —C(CH3)=CH—, and wherein B is —CH3, and p is 1, 2 or 3, and wherein the nanoemulsion forms a droplet with a diameter in the range of about 43 nm to about 96 nm.

According to another embodiment of the invention, a cosmetic composition may comprise the aforementioned nanoemulsion.

According to another embodiment, a method of treating skin comprises providing the aforementioned nanoemulsion; and applying the nanoemulsion to skin.

According to another embodiment of the invention, a nanoemulsion comprises a compound represented by Formula 1:

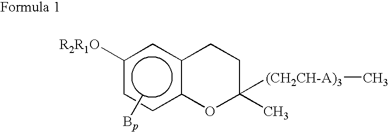

wherein R₁ is —(O—CH₂—CH₂)ₘ—, m is an integer from 0 to 300, wherein R₂ is H—(O—CH(CH₃)—CH₂)ₙ—, n is an integer from 1 to 250, wherein A is —CH₂—CH(CH₃)— or —C(CH3)=CH—, and wherein B is —CH3, and p is 1, 2 or 3.

According to another embodiment, a cosmetic composition may comprise the aforementioned nanoemulsion.

According to another embodiment, a method of treating skin may comprise: providing the nanoemulsion of Claim 23; and applying the nanoemulsion to skin.

### BRIEF DESCRIPTION OF THE DRAWINGS

FIG. 1 shows a Cryogenic-Transmission Electron Microscope (Cryo-TEM) microphotograph of a nanoemulsion cosmetic composition according to an embodiment of the invention, 1 day after preparation.

FIG. 2 shows a Cryo-TEM microphotograph of a nanoemulsion cosmetic composition according to another embodiment, 1 day after preparation.

FIG. 3 shows an Atomic Force Microscope (AFM) microphotograph of a nanoemulsion cosmetic composition according to an embodiment of the invention, 1 day after preparation.

FIG. 4 shows an AFM microphotograph of a nanoemulsion cosmetic composition according to another embodiment, 1 day after preparation.

### DETAILED DESCRIPTION OF EMBODIMENTS

On recognizing the importance of nanoemulsions in the industries of cosmetics, drugs, etc. a method of preparing nanoemulsions using POP-POE vitamin as an emulsifier was proposed, as disclosed in Korea Patent No. 488220, and incorporated by reference in its entirety herein. The method discloses the preparation of nanoemulsions not only of low viscosity but also of high viscosity or hardness by simple stirring using a stirrer or a homogenizer at 1 atmospheric pressure. This preparation method is different from the difficult and inefficient conventional nanoemulsification methods of using a high-pressure homogenizer or a PIT principle. In the nanoemulsification method described above, stable nanoemulsions can be prepared economically by the “2 phase complex emulsification method” which consists of two steps of forming a high viscosity complex of an oil soluble phase and a water soluble phase of nanoemulsions. The high viscosity complex is formed by mixing and heating, and then emulsifying the high viscosity complex by a high speed propeller mixer.

The nanoemulsification method disclosed in Korea Patent No. 488220 was more economical than those methods using a high-pressure homogenizer or the PIT principle and prepared excellent nanoemulsions having a mean droplet diameter in the range of about 206 nm to about 455 nm. The polyoxypropylene-polyoxyethylene vitamin E compound disclosed in Korea Patent No. 488220 is described in Formula 1:

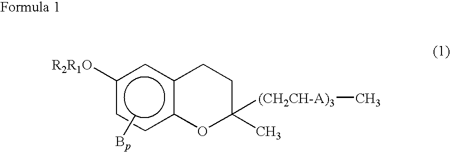

wherein, R1 is —(O—CH₂CH₂)ₘ—, m is an integer of 0 to 300, R2 is H(OCH(CH₃)CH₂)ₙ— and n is an integer from 1 to 250, A is

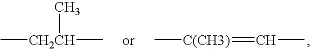

B is —CH3 at the 5-, 7- or 8-position of vitamin E, and p is an integer from 1 to 3.

The principles of forming nanoemulsions according to embodiments of the invention are as follows.

The ratio of the depth of adsorption layer (δ) of the emulsifier to the diameter of droplet (R) in the nanoemulsion with a smaller droplet diameter is larger than that with a larger droplet diameter. Therefore, the repulsive force is very large and prevents the aggregation of the systems. For droplets to be stable, complete coverage of droplet surfaces is required. If this condition is not fulfilled the dispersion is destabilized. If the dispersed droplets are coated partially with polymeric emulsifiers and the molecular weight of polymers is large enough, the polymeric emulsifiers can bind up the two droplets coated with polymeric emulsifiers, i.e. polymeric emulsifiers make a bridge and cause flocculation. But at high shear rates the large flocculated droplets are disentangled and dispersed in equal distance. If the distance between droplets becomes close to any value, the dispersion system shows a rapid increase in viscosity and the viscoelastic behavior of the system becomes dependent on the ratio of δ to R. The reduction in δ with increasing volume or weight fraction of the dispersed phase (Φ) may be attributed to the interpenetration and/or compression of chains with increasing Φ. The reduction in δ value can also be attributed to the compression of chains on close approach without the need to invoke any interpenetration.

Thus if the droplet size is small, not only aggregation or coalescence but precipitation is also prevented. Polymeric emulsifiers give steric stability and help active components transport into the skin effectively because of the favorable absorption properties. But these properties are the main reasons for why nanoemulsions cannot be prepared by the conventional method of using a high pressure homogenizer (500-1600 atmospheric pressure) for o/w nanoemulsions having more than 20% of inner oil phase in general. If the distance between droplets becomes less than the critical distance by high pressure the droplets become aggregated easily and heterogeneous. Therefore, novel nanoemulsification methods other than using a high pressure homogenizer are desirable for preparing nanoemulsions of higher quality.

Steric stabilization is generally dependent on the flocculation of sterically stabilized dispersion. The Θ-point is the temperature at which interaction between polymers and solvent is just as high as that between segments of the polymers. But in embodiments of the invention, the Θ-point is defined as the temperature at which a viscoelastic complex of high viscosity or hardness is formed by the interaction of oil phase and water phase.

The Θ-point is determined by the measurement of the viscoelastic property of emulsion systems on heating and stirring slowly. In order to prevent the coupling of the formed droplets an energy barrier should be formed. When a stabilizing agent is added to the emulsion systems an energy barrier is formed wherein a boundary of mono molecules or multi-layer liquid crystals are formed. For the systems to be sterically stable interaction between droplets should be large enough. For these reasons good polymeric emulsifiers should have tails dissolved in the outer phase with segments insoluble in the outer phase and segments adhered to the droplets in the inner phase, contributing to the stability of the emulsion system.

When linear polymers are put into solvent molecules they penetrate into vacant areas formed by the decreased mutual interaction between polymer molecules and the non-crystal areas first and gradually penetrate into crystal areas of high density. Therefore, in the beginning, parts of the linear polymers become swollen by coupling with the crystal area or by entanglement. Dissolution is obtained when this coupling is decoupled and molecular segments are free to move. When polymer ends are combined to any other than ends the reaction is called a cross linking reaction and the branched part is called a crosslink. If some parts of molecular chains are bound by crosslinking, each molecular chain is not separated and can not be swollen to a constant size. The crosslinking reaction occurs by the proper monomers or occurs by several reactions after polymerization. The crosslinking reactions make crosspolymers of different degrees of crosslinking by the crosslinking method and conditions applied. Crosslinking polymers of low degrees or high degrees can be obtained by controlling the number of crosslinks in the definite length of chains. Fewer crosslinked polymers have a good recovering (elastic) property as described above. In any swollen state with remnant network structures formed by the cross-links, liquid crystal phase or entanglement is called a gel. Gels are divided into viscoelastic gels and non viscoelastic gels.

Given the above, o/w nanoemulsion cosmetic compositions containing polymeric emulsifier POP-POE vitamin E with a mean droplet diameter of 43-96 nm and with good productivity and safety have been obtained to improve upon the nanoemulsion compositions containing polymeric emulsifier POP-POE vitamin E with a mean droplet diameter of 206-455 nm, as disclosed in Korea Patent No. 488220. The methods of preparing the nanoemulsions comprise using a polyacrylic acid crosspolymer or a polyacrylic acid derivative crosspolymer as an assistant emulsifier. Although the polyacrylic acid crosspolymer and the polyacrylic acid derivative crosspolymer themselves have poor emulsifying abilities, they have good gel-forming properties along with emulsifiers such as PO-POE vitamin E at the Θ-point above 40□, preferably above 50□, through energy transferring systems of high efficiency such as high-speed stirring that can absorb rotational frictional energy

The nanoemulsions prepared with emulsifiers, POP-POE vitamin E, along with emulsion assistants, polyacrylic acid crosspolymers or polyacrylic acid derivative crosspolymers by high-speed stirring according to embodiments of the invention showed much improved properties in the formation of viscoelastic complexes of oil soluble phase and water soluble phase with high viscosity or hardness. That is, nanoemulsion according to embodiments of the invention showed improved emulsification properties than those prepared only with emulsifiers, POP-POE vitamin E, due to the improved abilities for absorbing rotational frictional energy necessary for emulsification to fine droplets.

The emulsifiers in nanoemulsion compositions in embodiments of the invention are POP-POE vitamin E as described in general formula 1 wherein m is preferably an integer from 0 to 300 and m is an integer from 1 to 250, more preferably m is an integer from 20 to 150 and n is an integer from 10 to 100. POP-POE vitamin E in general formula 1 can be prepared by the preparation method disclosed in Korea Patent publication No. 2000-0000840 or commercially obtained.

The polyacrylic acid crosspolymers or polyacrylic acid derivative crosspolymers are homopolymers, Carbomers, obtained by the cross linking reactions of a polyacrylic acid chain of Formula 2 below with allylether of polyol or allylether of propylene or copolymers, acrylates/C₆-C₄₀ alkylacrylate crosspolymers prepared by the crosslinking reaction of C₆-C₄₀ alkylacrylates or more than 1 acrylic acid or methacrylic acid or their esters with allylether of polyol. In this invention, unlimited examples of allylethers of polyol could be used, but allylether of pentaerythritol or allylether of sucrose are suggested.

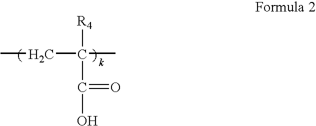

wherein R₄ is H or CH₃, k is an integer of 10 to 100,000.

Emulsion assistants, polyacrylic acids or their derivative crosspolymers are manufactured using the method described above or are commercially obtained. Of carbomers, product names that are commercially available in the markets are, for example, Carbopol 910, -934, -940, -934p, -954, -961, -980, -9890, -981, -2984, -5984, -ETD2001, ETD2050, Carbopol Ultretz 10 of Goodrich company, Acritamer 501E, -504E, -934, -943, -941 of Rita. Of acrylates/C₆-C₄₀ alkylacrylate crosspolymers, commercial products are, for example, Carbopol 1342, -1382, ETD 2020, Pemulen-TR-1, -TR-2 of Goodrich company.

The POP-POE vitamin E in the nanoemulsion cosmetic compositions according to embodiments of the invention comprises 0.5 to 60% weight fraction, preferably 1.0 to 30% weight fraction, of the total nanoemulsion.

When POP-POE vitamin E is used as an emulsifier, the emulsifiers are adsorbed easily at the interface of oil soluble phase and water soluble phase by the easy formation of liquid crystals, resulting in the favorable formation of complex of oil soluble phase and water soluble phase, which makes smaller oil-soluble phase droplets by stirring.

According to embodiments of the invention, the diameter of the nanoemulsion droplet is for example, about 300 nm, about 295 nm, about 290 nm, about 285 nm, about 280 nm, about 275 nm, about 270 nm, about 265 nm, about 260 nm, about 255 nm, about 250 nm, about 245 nm, about 240 nm, about 235 nm, about 230 nm, about 225 nm, about 220 nm, about 215 nm, about 210 nm, about 205 nm, about 200 nm, about 195 nm, about 190 nm, about 185 nm, about 180 nm, about 175 nm, about 170 nm, about 165 nm, about 160 nm, about 155 nm, about 150 nm, about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, about 80 nm, about 75 nm, about 70 nm, about 65 nm, about 60 nm, about 55 nm, about 50 nm, about 45 nm, about 40 nm, about 35 nm, about 30 nm, about 25 nm, about 20 nm, about 15 nm, about 10 nm, or about 5 nm. According to embodiments of the invention, the diameter of the nanoemulsion droplet is in a range formed by two of the numbers in the foregoing sentence.

The o/w nanoemulsion cosmetic compositions according to embodiments of the invention are safe because the use of polymeric emulsifiers are limited in their penetration into the skin. According to embodiments of the invention the skin protecting agents and skin treating agents in the o/w nanoemulsion cosmetic compositions show more improved effects of skin protection and skin treatment than in emulsion compositions, due to the properties of nanoemulsions, such as their stability and excellent skin penetration activity.

The emulsion assistants, polyacrylic acid or its derivative crosspolymers, of the nanoemulsion cosmetic compositions according to embodiments of the invention comprise 0.01 to 40% weight fraction, preferably 0.02 to 10% weight fraction, of the total nanoemulsion.

According to embodiments of the invention, along with POP-POE vitamin E as emulsifiers, and polyacrylic acid or its derivative crosspolymers as emulsion assistants, components of o/w nanoemulsion cosmetic compositions include oil-soluble components and water soluble components being generally used to form products that protect skin in cosmetic compositions. The representative oil soluble materials are as follows: hydrocarbon materials of paraffin oil, squalene; synthetic triglycerides such as caprylic/capric triglyceride (Neobee M-5) obtained from the reaction of natural material; synthetic ester oils such as cetyloctanoate, octyldodecanol (Eutanol G), isopropyl palmitate; plant oils such as jojoba oil, olive oil, safflower oil, evening primrose oil, Chinese pepper oil, sesame oil; shark oil; oil soluble vitamins such as vitamin A, E, F and their derivatives and representative water soluble materials as follows; polyols such as propylene glycol, butylene glycol, glycerine, polyethylene glycol; mucopolysaccharides such as hyaluronic acid, condroitin sulfate, glucosamine, water-soluble vitamins and their derivatives such as vitamin C and panthenol.

Nanoemulsion cosmetic compositions were prepared as follows. A first emulsification step was carried out at about 0.4 to 0.75 weight ratio of oil soluble phase to oil soluble plus water soluble phase. A second emulsification step was carried out after adding all remnant materials of the oil soluble phase and water soluble phase that have not emulsified.

For stability and feel when applied on the skin, polymers other than the emulsion assistants, such as polyvinyl pyrolidone, methylcellulose, hydroxymethylcellulose (Natrosol 250HR), and thickeners such as magnesium aluminium silicate (Veegum HV), sodium aluminium silicate (Laponite XLG), are preferably used individually or together with embodiments of the invention. The additions can have a content of 0.01 to 40% by weight fraction, but more preferably 0.05 to 20% by weight fraction.

Additives of o/w nanoemulsion cosmetic compositions in embodiments of the invention are used as much as 0.1 to 30% by weight of total compositions according to the purpose of the products. The additives can include the following: ultra violet ray absorbers of benzophenone derivatives such as homomethylsalisilates, benzophenone, 2-hydroxybenzophenone, 4-methoxybenzophenone; cinnamic acid derivatives such as ethylhexyl-p-methoxy cinnamate, octylmethoxycinamate, butylmethoxydibenzophenone; skin whitening agents such as arbutin, kojic acid, uvaursi extract, etc.; skin cell circulation promoting agents such as alpha-hydroxy acid, pancreatin, good skin protecting agents such as allantoin, amino acid, protein, flavonoids, milk, honey; skin protecting natural plant extracts such as extracts of angelica acutiloba, cnidium officinale, Chinese bellflower, calendula arvensis, ginseng, green tea; skin coloring dihydroxyacetone and zinc oxide having a skin soothing effect. Additionally, components of skin protecting agents and skin treating agents of nanoemulsion drugs for curing skin diseases can also be included. For example, they can include: hydroquinone skin depigmentation agents; agents accelerating the turnover of corneous layers, such as salicylic acid, alpha-hydroxy acid, sulfur and enzyme pancreatin in acne skin curing products; and skin disease curing agents such as hydrocortisone, a component of adrenaline hormones. The components described above can be used without any limitations and these examples do not limit the use of any other components which can be used generally in products for skin protection and skin disease treatments.

O/w nanoemulsion cosmetic compositions in embodiments of the invention can be classified into cream products and lotion products. The former have no fluidity and the latter have fluidity at room temperature. These products can be used according to the added active materials as several kinds of skin protecting products and skin disease curing products such as skin care cosmetics; moisture creams and lotions, night creams, eye creams, cleansing creams and lotions, sunscreen creams and lotions, suntanning creams and lotions, skin whitening creams & lotions, anti-wrinkle creams and lotions, acne care creams and lotions and atopy care creams and lotions and dispersion type make-up cosmetics; foundation, make-up base and hair care cosmetics of creams and lotions; hair dye, hair treatment cream, hair cream and skin curing topical drugs of creams and lotions; skin depigmentation products, anti-wrinkle products, acne curing products and adrenalin hormone products.

o/w nanoemulsion cosmetic compositions in embodiments of the invention are prepared characteristically only under the condition that the complex of oil soluble phase and water soluble phase becomes highly viscous or hard at θ-point above 40□. The nanoemulsion in embodiments of the invention can be prepared in an economical way due to the efficient energy transfer of the high viscosity complex of oil-soluble phase and water soluble phase along with emulsifiers and emulsion assistants at θ-point, wherein the viscosity of the complex was not decreased by heating but instead was maintained or increased.

In the preparation of o/w nanoemulsion cosmetic compositions in embodiments of the invention, high speed rotational mixers such as propellers, dispersers, and homogenizers generating enough frictional energy can be used for forming the high-viscosity complex of oil soluble components and water soluble components above 40□ of the Θ-point.

Embodiments of the invention will be described further through the examples below. But these examples are only suggestions for embodiments of the invention and the range of values is not restricted to those disclosed below.

### Examples 1-4 and Comparative Examples 1-4

### O/W Nanoemulsion Cosmetic Base Compositions

According to the compositions in Table 1 oil soluble materials and emulsifiers were poured into the manufacturing tank and heated to 50° C. followed by adding water soluble materials previously heated and dispersed. After confirming the occurrence of the Θ-point of the mixture by slow heating and stirring with a propeller mixer, the o/w nanoemulsion base compositions in Examples 1-4 were prepared by emulsifying by a high speed mixer (1,000-8,000 RPM) at 1 atmospheric pressure and cooling to 30° C.

Comparative Examples 1-4 in Table 1 were prepared by the same method employed in Examples 1-4.

TABLE 1

| | | | | | | Comparative | | | |
|---|---|---|---|---|---|---|---|---|---|
| | | Examples (%) by | | | | Example (%) by | | | |
| | | Weight | | | | Weight | | | |
| Function | Component | 1 | 2 | 3 | 4 | 1 | 2 | 3 | 4 |
| A. Oil soluble phase | Liquid paraffin | 30 | 40 | 30 | 40 | 30 | 40 | 30 | 40 |
| | Neobee M-5 | 10 | 20 | 10 | 20 | 10 | 20 | 10 | 20 |
| | Eutanol G | 10 | 10 | 10 | 10 | 10 | 10 | 10 | 10 |
| | Preservative | Q.S. | Q.S. | Q.S. | Q.S. | Q.S. | Q.S. | Q.S. | Q.S. |
| B. Emulsifier | POP(20)-POE(50) | 2 | 2 | 4 | 4 | 2 | 2 | 4 | 4 |
| | vitamin E | | | | | | | | |
| | Butylene glycol | 3 | 3 | 3 | 3 | 3 | 3 | 3 | 3 |
| | Carbopol 941 | 0.1 | — | — | — | — | — | — | — |
| | Carbopol 940 | — | 0.1 | — | — | — | — | — | — |
| C. Water soluble | Pemulen TR-1 | — | — | 0.1 | — | — | — | — | — |
| phase | Pemulen TR-2 | — | — | — | 0.1 | — | — | — | — |
| | Triethanolamine | 0.1 | 0.1 | 0.1 | 0.1 | — | — | — | — |
| | Deionized water | to 100 | to 100 | to 100 | to 100 | to 100 | to 100 | to 100 | to 100 |

| | | | | | | | | |
|---|---|---|---|---|---|---|---|---|
| Φ₀(weight): [oil soluble phase | 0.51 | 0.71 | 0.52 | 0.73 | 0.51 | 0.71 | 0.52 | 0.73 |
| (oil-soluble phase + water soluble | | | | | | | | |
| phase)] | | | | | | | | |
| Θ-Point, ° C. | 63 | 67 | 65 | 75 | 60 | 65 | 65 | 75 |

### Experimental Example 1

### Evaluation of Nanoemulsion Stability by Measuring Droplet Sizes and the Changes of Droplet Sizes

The droplet sizes of o/w nanoemulsion cosmetic base compositions prepared according to Examples 1-4 and Comparative Examples 1-4 were measured by the dynamic light scattering method using the Master sizer 2000 (Malvern Instrument, U.K.). The measurements of droplet sizes were carried out after diluting emulsions with deionized water to 10-20% obscuration under the following conditions. The test results are shown in Table 2.

Measuring time: 2 min; measuring times per second: 5×10³; temperature: 20° C.; viscosity: 0.89 centipoise; diffraction rate of particles: 4; diffraction rate of the dispersion phase: 1.33.

Thermodynamic stability of nanoemulsions was evaluated by measuring the droplet size of nanoemulsions two times, 1 day after preparing and 6 months after storing at 40° C.

### Experimental Example 2

### Emulsion Stability by Visual Observation

The stability of o/w nanoemulsion cosmetic base compositions obtained in Examples 1-4 above was evaluated by visual observation together with droplet size measurements according to the following procedure. The test results are shown in Table 2.

Emulsion states of compositions in Examples 1-4 and Comparative Examples 1-4 were comparatively observed both 1 day after preparation and 6 months after storing at 40° C. The instabilities such as sedimentation, separation, drain, creaming and coalescence were visually observed.

Stability of emulsions is evaluated by the percentage of stable portion of the total portion and is expressed by the following equation.

Emulsion stability (%)=(Total portion-instable portion)/total portion)×100%

TABLE 2

| | Droplet size (nm) | | | Emulsion |
|---|---|---|---|---|
| | | 6 months | | stability |
| Formulation | 1 day after | after storing | Droplet size | by visual |
| No. | preparation | at 40° C. | Increase (%) | observation |
| Example 1 | 73 | 77 | 5.5 | 94 |
| Example 2 | 85 | 90 | 5.9 | 96 |
| Example 3 | 52 | 53 | 3.8 | 96 |
| Example 4 | 48 | 50 | 4.2 | 98 |
| Comparative | 253 | 275 | 9.7 | 86 |
| Example 1 | | | | |
| Comparative | 326 | 352 | 7.9 | 85 |
| Example 2 | | | | |
| Comparative | 205 | 223 | 8.8 | 90 |
| Example 3 | | | | |
| Comparative | 248 | 267 | 7.7 | 90 |
| Example 4 | | | | |

As shown in Table 2, the mean droplet diameter of o/w nanoemulsion cosmetic base compositions in Examples 1-4 prepared by the emulsification method of simple mixing using a propeller mixer at 1 atmospheric pressure was in the range of 48 rim to 85 nm 1 day after preparation, which is a very close value to the minimum droplet diameter of nanoemulsions, 20 rim, and much smaller than those in Comparative Examples 1-4. And in the evaluation of stability by measuring the droplet size 6 months after storing at 40° C. the mean droplet diameter increase (%) in Examples 1-4 was about 4.8%, which is smaller than that of Comparative Examples 1-4, 8.5%.

Table 2 also shows the stability (%) of o/w nanoemulsion cosmetic base compositions in Examples 1-4 by visual observation 6 months after storing at 40° C. As shown in Table 2 above, the compositions in Examples 1-4 were more stable than those in Comparative Examples 1-4.

### Experimental Example 3

### Human Patch Test

In order to confirm the safety of cosmetic compositions in Examples 1-4 and those in Comparative Examples 1-4, a human patch test was carried out. Confirmation of the safety of nanoemulsions is considered important since skin irritation problems occurred often with conventional nanoemulsions because of using relatively a high amount of emulsifier phospholipids to obtain a smaller droplet. The Finn chamber human patch test for o/w nanoemulsion cosmetic base compositions of Examples 1-4 was carried out, and the test results are shown in Table 3.

As shown in Table 3, o/w nanoemulsion cosmetic base compositions in Examples 1-4 were safer than those in Comparative Examples 1-4.

TABLE 3

| | Irritation rate (%) (n = 50) | | |
|---|---|---|---|
| Composition | after 4 hr | after 48 hr | mean (%) |
| Example 1 | 0 | 0 | 0 |
| Example 2 | 0 | 0 | 0 |
| Example 3 | 0.06 | 0 | 0.03 |
| Example 4 | 0.06 | 0 | 0.03 |
| Comparative | 0 | 0 | 0 |
| Example 1 | | | |
| Comparative | 0.04 | 0 | 0.02 |
| Example 2 | | | |
| Comparative | 0.4 | 0.06 | 0.08 |
| Example 3 | | | |
| Comparative | 0.1 | 0.04 | 0.07 |
| Example 4 | | | |

### Example 5 and Comparative Examples 5-1˜5-5

### O/W Nanoemulsion Cosmetic Base Compositions

According to the compositions in Table 4, oil soluble phase and emulsifier were poured into the manufacturing tank followed by heating them to 50° C. and the dispersed water soluble phase preheated to 50° C. was added. The o/w nanoemulsion cosmetic base composition in Example 5 was prepared by the emulsification method using a high speed disperser mixer (1,000-8,000 RPM) at 1 atmospheric pressure after confirming the formation of a high viscosity complex at the Θ-point by stirring slowly upon heating the mixtures of oil soluble phase with emulsifier and water-soluble phase, and then cooling down to RT. Those of Comparative Examples 5-1, -2, -3, -4, -5 in Table 4 were prepared by the same method employed in Example 5.

TABLE 4

| | | Example | Comparative Example | | | | |
|---|---|---|---|---|---|---|---|
| | | (% by weight) | (% by weight) | | | | |
| Function | Component | 5 | 5-1 | 5-2 | 5-3 | 5-4 | 5-5 |
| A. Oil soluble | Cetostearyl | 3 | 3 | 3 | 3 | 3 | 3 |
| phase | alcohol | | | | | | |
| | Paraffin | 5 | 5 | 5 | 5 | 5 | 5 |
| | Paraffin oil | 40 | 40 | 40 | 40 | 40 | 40 |
| | Neobee M5 | 15 | 15 | 15 | 15 | 15 | 15 |
| | Eutanol G | 5 | 5 | 5 | 5 | 5 | 5 |
| | Preservative | Q.S. | Q.S. | Q.S. | Q.S. | Q.S. | Q.S. |
| B. Emulsifier | POP(20)-POE(50) | 3 | 3 | 3 | — | — | — |
| | vitamin E | | | | | | |
| | POE(40) Vitamin E | — | — | — | 3 | — | — |
| | Soybean | — | — | — | — | 3 | — |
| | phospholipid | | | | | | |
| | Polysorbate-60/ | —/— | —/— | —/— | —/— | —/— | 1.7/ |
| | Sesquioleate | | | | | | 0.3 |
| C. Water soluble | Butylene glycol | 3 | 3 | 3 | 3 | 3 | 3 |
| phase | Carbopol 941 | 0.12 | — | — | 0.06 | 0.06 | 0.06 |
| | Natrosol 250HR | — | — | 0.2 | — | — | — |
| | Triethanolamine | 0.1 | — | — | 0.1 | 0.1 | 0.1 |
| | Deionized water | to 100 | to 100 | to 100 | to 100 | to 100 | to 100 |

| | | | | | | |
|---|---|---|---|---|---|---|
| φ₀(weight) [Oil soluble phase/ | 0.72 | 0.72 | 0.72 | 0.72 | 0.72 | 0.72 |
| (oil soluble phase + water soluble | | | | | | |
| phase)] | | | | | | |
| θ-Point, ° C. | 70 | 72 | 68 | — | — | — |

### Experimental Example 5

### Evaluation of Nanoemulsion by Measuring the Droplet Sizes and the Changes of Droplet Sizes

The droplet sizes of o/w nanoemulsion cosmetic base compositions prepared according to Example 5 and Comparative Examples 5-1 to 5-5 were measured by the same method employed in Experimental Example 1-4 in Example 1-4, and the test result 1 day after and 6 months after storing at 40° C. are shown in Table 5.

TABLE 5

Droplet size, nm

| | | 6 months | |
|---|---|---|---|
| Formulation | 1 day after | after storing | Droplet size |
| No. | storing at RT | at 40° C. | Increase (%) |
| Example 5 | 45 | 46 | 2.2 |
| Comparative | 228 | 248 | 10.0 |
| Example 5-1 | | | |
| comparative | 241 | 258 | 7.1 |
| Example 5-2 | | | |
| Comparative | — | — | — |
| Example 5-3 | | | |
| Comparative | — | — | — |
| Example 5-4 | | | |
| Comparative | — | — | — |
| Example 5-5 | | | |

The “−” sign in Table 5 means that the composition was not emulsified by the emulsification method employed in embodiments of the invention.

As shown in Table 5 the mean droplet diameters of o/w nanoemulsion cosmetic base composition of Example 5 prepared by the simple rotational mixing method using a disperser mixer at 1 atmospheric pressure 1 day after preparation at room temperature (RT) was 45 nm, which is close to the minimum value of the nanoemulsion droplet diameter range, 20-500 nm, but that of Comparative Examples 5-1 and 5-2 was 235 nm. On the other hand, nanoemulsions were not prepared when the base compositions of Comparative Examples 5-3, 5-4 and 5-5 were emulsified by the same nanoemulsification method using a simple rotational mixer employed in Example 5. Namely, those compositions did not form the high viscosity complex when the oil soluble phase was mixed with the water soluble phase along with an emulsifier and an emulsion assistant, carbopol, i.e., the mixture did not show a 0-point. The mean droplet diameter of the compositions of Comparative Examples 5-3, 5-4 and 5-5 were 1.675 nm, 2.278 nm and 3.258 nm, respectively, when prepared by the emulsification method using a homogenizer. Increase (%) in the mean droplet diameters of composition in Example 5 by evaluating the droplet diameters of the emulsion compositions 6 months after storing at 40° C. was 2.2%, which is much smaller than that of Comparative Examples 5-1 and 5-2, 8.6%.

### Examples 6-7 and Comparative Examples 6-7

### O/W Nanoemulsion Cosmetic Cream Compositions

The purpose of Examples 6-7 is to confirm the effect of additives in preparing nanoemulsions, since the property of nanoemulsions can be affected by the additives even though the droplet diameter of emulsion or nanoemulsion is mainly dependent on the base components.

According to the compositions in Table 1, oil soluble materials and emulsifiers were poured into the manufacturing tank and heated to 50° C. followed by adding water soluble materials previously heated to 50° C. and then dispersed. After confirming the occurrence of the O-point of the mixture by slow heating and stirring using a propeller mixer, the o/w nanoemulsion base compositions in Examples 1-4 were prepared by emulsifying using a high speed mixer (1,000-8,000 RPM) at 1 atmospheric pressure and then cooling to 30° C.

Comparative Examples 1-1 in Table 1 were manufactured by the same method for comparison.

According to the compositions in Table 6, oil-soluble raw materials 1-11 were poured into a manufacturing tank followed by melting on heating to 70° C. The tank was kept after adding raw materials 12-14. Next, water soluble raw materials 18-29 dissolved in a supplementary tank at 50° C. were added to the manufacturing tank. Afterwards, the high viscosity 2 phase complex of oil soluble phase and water soluble phase were prepared by slowly mixing using a homogenizer on increasing the temperature to Θ-point. The o/w nanoemulsion moisture cream was prepared by cooling homogenized emulsions to 30° C., which had been subjected previously to stirring again using a disperser (1,000-8,000 RPM) after cooling to 50° C. and adding oil soluble materials 15-17 to the high viscosity complex at 1 atmospheric pressure.

TABLE 6

| | | | | Comparative | Comparative |
|---|---|---|---|---|---|
| | | Example 6 | Example 7 | Example 6 | Example 7 |
| Components | | 6 | 7 | 6 | 7 |
| 1 | Microcrystalline wax | 3.0 | 3.0 | 3.0 | 3.0 |
| 2 | Paraffin | 2.0 | 2.0 | 2.0 | 2.0 |
| 3 | Bees wax | 3.0 | 3.0 | 3.0 | 3.0 |
| 4 | Cetostearyl alcohol | 2.0 | 2.0 | 2.0 | 2.0 |
| 5 | Glycerylmonostearate | 2.0 | 2.0 | 2.0 | 2.0 |
| 6 | Liquid paraffin | 25.0 | 25.0 | 25.0 | 25.0 |
| 7 | Neobee M5 | 18.0 | 18.0 | 18.0 | 18.0 |
| 8 | Eutanol G | 5.0 | 5.0 | 5.0 | 5.0 |
| 9 | Evening primrose oil | 0.5 | 0.5 | 0.5 | 0.5 |
| 10 | Preservative | Q.S. | Q.S. | Q.S. | Q.S. |
| 11 | Antioxidant | Q.S. | Q.S. | Q.S. | Q.S. |
| 12 | POP(30)-POE(70) vitamin E | 3.0 | 2.5 | 3.0 | 2.5 |
| 13 | POP(10)-POE(30) vitamin E | 0.5 | 0.5 | 0.5 | 0.5 |
| 14 | POP(70)-POE(150) vitamin E | 0.5 | 0.5 | 0.5 | 0.5 |
| 15 | Vitamin A palmitate | 0.2 | 0.2 | 0.2 | 0.2 |
| 16 | Vitamin E acetate | 0.5 | 0.5 | 0.5 | 0.5 |
| 17 | Perfume | Q.S. | Q.S. | Q.S. | Q.S. |
| 18 | Glycerine | 6.0 | 6.0 | 6.0 | 6.0 |
| 19 | Butylene glycol | 4.0 | 4.0 | 4.0 | 4.0 |
| 20 | Allantoin | 0.1 | 0.1 | 0.1 | 0.1 |
| 21 | Hyaluronic acid | 1.0 | 1.0 | 1.0 | 1.0 |
| 22 | Sequestering agent | Q.S. | Q.S. | Q.S. | Q.S. |
| 23 | Bearberry extract | 1.0 | 1.0 | 1.0 | 1.0 |
| 24 | Angelica actutiloba extract | 5.0 | 5.0 | 5.0 | 5.0 |
| 25 | Cnidium officinale extract | 3.0 | 3.0 | 3.0 | 3.0 |
| 26 | Carbopol 940 | 0.1 | 0.06 | — | — |
| 27 | Natrosol 250HR | — | — | 0.2 | — |
| 28 | Triethanolamine | 0.1 | 0.1 | — | — |
| 29 | Deionized water | to 100 | to 100 | to 100 | to 100 |

| | | | | |
|---|---|---|---|---|
| Φ₀(Weight) [Oil soluble phase/(Oil | 0.63 | 0.62 | 0.63 | 0.62 |
| soluble phase + Water soluble | | | | |
| phase)] | | | | |
| Θ-Point, ° C. | 68 | 72 | 70 | 72 |

### Experimental Examples 6-7

### Evaluation of Nanoemulsion Stability by Measuring the Droplet Sizes and the Changes of Droplet Sizes

For evaluating the nanoemulsion stability of o/w nanoemulsion cosmetic moisture cream compositions obtained in Examples 6-7 and Comparative Examples 6-7 the mean droplet sizes of those compositions were measured by the same method employed in Experimental Example 1 of Examples 1-4.

The test result of the mean droplet diameters of o/w nanoemulsion moisture cream compositions of Examples 6-7 and Comparative Examples 6-7 in Table 6, 1 day after preparation and 6 months after storing at 40° C., are shown in Table 7.

TABLE 7

Droplet size, nm

| | 1 day | 6 months | |
|---|---|---|---|
| Formulation | after storing | after storing | Droplet size |
| No. | preparation | at 40° C. | increase (%) |
| Example 6 | 48 | 46 | 4.3 |
| Example 7 | 51 | 50 | 0.0 |
| Comparative | 231 | 241 | 4.3 |
| Example 6 | | | |
| Comparative | 215 | 230 | 7.0 |
| Example 7 | | | |

As shown in Table 7 the mean droplet diameter of o/w nanoemulsion cosmetic compositions prepared by simple rotational mixing using a disperser mixer 1 day after preparation was in the range of 48 to 51 nm, which is a very close value to the minimum value of nanoemulsions, 20 nm.

On the other hand the mean droplet diameter of nanoemulsion compositions of Comparative Examples 6-7 was in the range of 215 to 231 nm, which is larger than that of Examples 6-7 by 182 nm. In the evaluation of stability by measuring the mean droplet diameter after storing for 6 months at 40° C. the increase (%) of the mean droplet diameter of compositions of Examples 6-7 was 1.1%, which is much smaller than that of Comparative Examples 6-7, 5.7%.

### Experimental Example 2

### Observation of Nanoemulsions by Cryogenic-Transmission Electron Microscope (Cryo-TEM)

Droplet sizes of emulsions in general can be measured by the indirect method of dynamic light scattering using a laser light. But in case of smaller nanoemulsions a direct measuring method along with an indirect measuring method provides more precise droplet size data for nanoemulsions. Observing fine droplets using a Cryo-TEM is a direct and precise method that can measure the droplet size without changing the state. Therefore, Cryo-TEM was used to confirm the mean droplet diameter of o/w nanoemulsion of Example 6, 45 nm, by the light scattering method, which is close to the minimum value of nanoemulsions, 20 nm. Cryo-TEM (CEM902A, Zeiss, D-Oberkochen, Philips, CM120, NL-Edinhoven) was operated both at 80 kV and 120 kV. An image of nanoemulsion droplets was acquired for the nanoemulsions diluted with deionized water, sonicated, applied on a copper grid as thinly as possible and frozen at 77-100° K for image treating systems (Kontron IBAS).

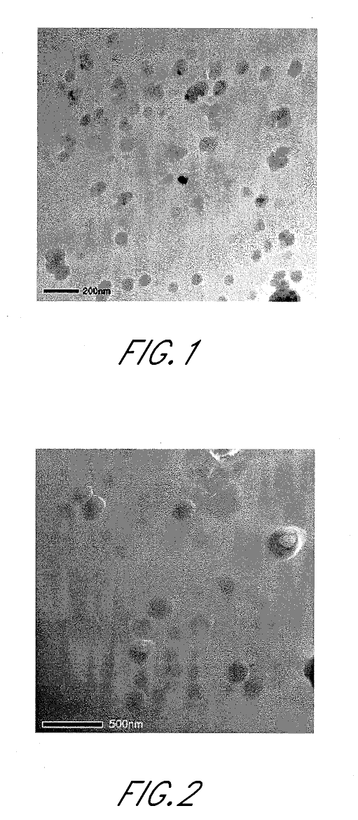

FIG. 1 is a microphotograph of Cryo-TEM for the nanoemulsion composition of Example 6, 1 day after preparation. FIG. 1 shows that the mean droplet diameter of o/w nanoemulsion cosmetic moisture cream of Example 6 is 50 nm, which is very similar to the value of 48 nm obtained by the dynamic light scattering method.

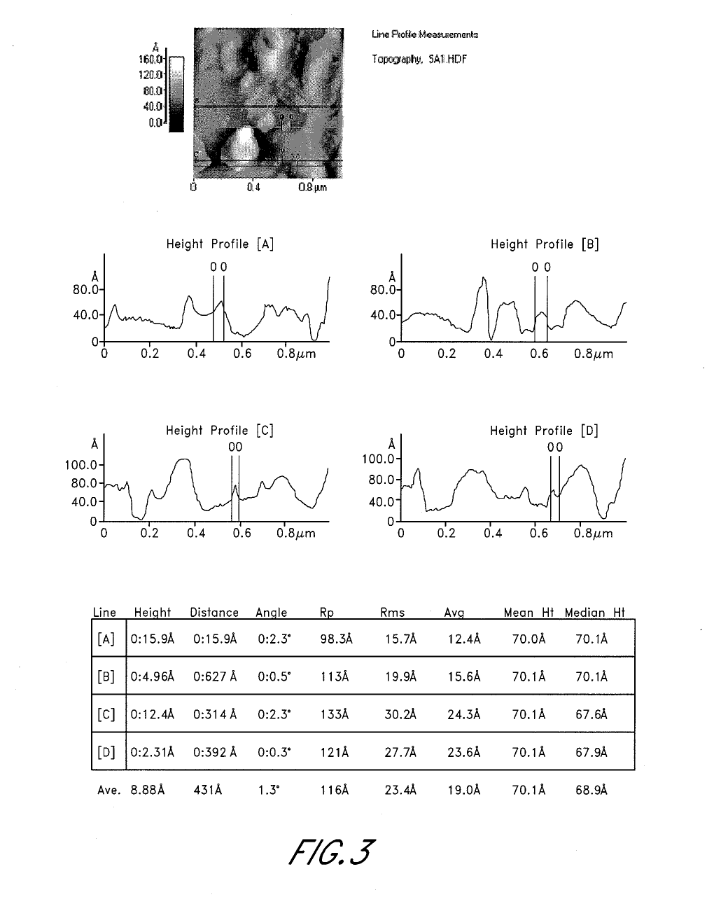

FIG. 2 is a microphotograph of nanoemulsions in Comparative Example 6, 1 day after preparation. FIG. 2 shows that the mean droplet diameter of nanoemulsion compositions of Comparative Example 6 is 210 nm, which is also a very similar value to 231 nm obtained by the dynamic light scattering method.

### Experimental Example 3

### Observation of Emulsion Droplets by Atomic Force Microscope (AFM)

Atomic force microscopes (AFM) are used for the morphological study of materials. The advantage of using this instrument is that the particles can be observed at RT and at 1 atmospheric pressure without any deformation of particles. In Experimental Example 3 an AFM was employed to observe the droplets of o/w nanoemulsion cosmetic moisture cream in Example 7.

The test sample was prepared using a pyramidal silicon nitride contilever with a force constant at 0.26 N/m. A photographic image was obtained with a non-contact scanning probe microscope (AutoProbe-CP: Park Scientific Instruments, CA, U.S.A.).

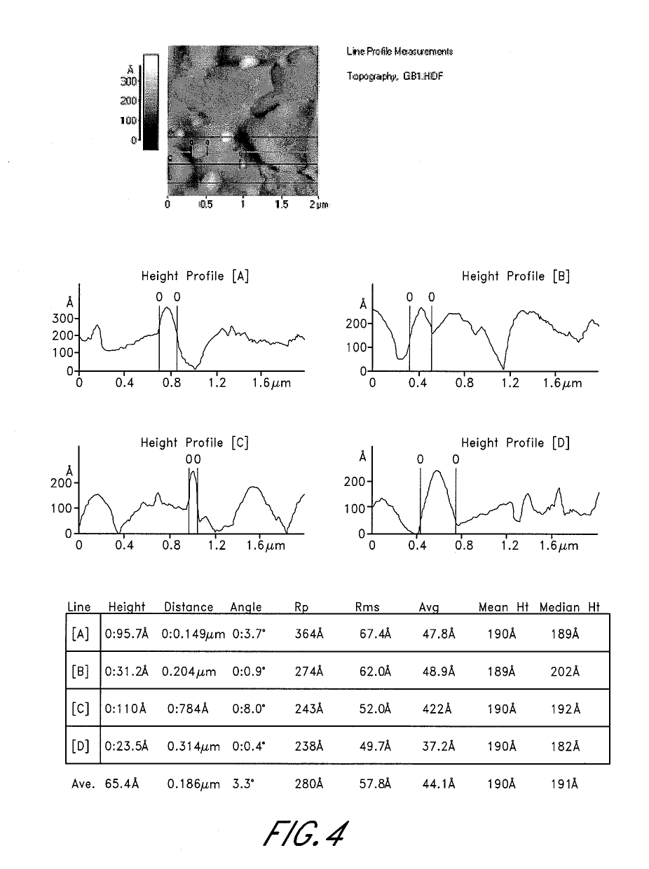

FIG. 3 is an AFM microphotograph of nanoemulsion cosmetic moisture cream in Example 71 day after preparation and FIG. 4 is that in Comparative Example 7, 1 day after preparation.

The mean droplet diameters of nanoemulsions in FIG. 3 and FIG. 4 are 43 nm and 186 nm, respectively.

### Experimental Example 4

### Product Effectiveness Test

To measure the effectiveness of o/w nanoemulsion cosmetic moisture cream compositions in Example 6 and Comparative Example 6, 4 groups of 80 women aged 20 to 50 were employed for a 3 month test. Among them 30 women had skin atopy with severe dryness and itching. The main goals for the test were to compare the effects of moisturizing, skin firming, skin wrinkle decreasing, whitening and skin atopy soothing, in order of priority. Other effects such as stickiness during application, spreadability on the skin, skin wetness and skin sheen were evaluated and given 4 grades of excellent, good, average and poor. The test results are shown in Table 8.

TABLE 8

| | | Comparative |
|---|---|---|
| Item/Product | Example 6 | Example 6 |
| Wrinkle decreasing effect | ◯◯◯ | ◯◯ |
| Whitening effect | ◯◯ | ◯◯ |
| Spreadability on skin | ◯◯◯ | ◯◯ |
| Atopy skin soothing effect | ◯◯◯ | ◯◯ |
| Smoothness | ◯◯◯ | ◯◯ |
| Moisturizing effect | ◯◯◯ | ◯◯ |
| Skin sheen | ◯◯◯ | ◯◯◯ |

◯◯◯: Excellent,
◯◯: Good,
◯: Average,
X: Poor

As shown in Table 8 o/w nanoemulsion cosmetic moisture cream in Example 6 showed better effects than that in Comparative Example 6 and particularly, the improved effect on itchy atopic skin caused from severe dryness was excellent.

### Examples 8-9 and Comparative Examples 8-9

### o/w Nanoemulsion Cosmetic Moisture Lotion Compositions

So far, cream o/w nanoemulsion cosmetic compositions and their preparation methods have been described through Examples 1-7 and Comparative Examples 1-7. Lotion o/w nanoemulsion cosmetic compositions or nanoemulsion lotion will be described next. The nanoemulsion cosmetic composition is generally defined as a cream when it has no fluidity, but a lotion when it has fluidity at RT.

O/w lotions have in general less amount of waxes with high melting points and oils as inner phase than creams and have a characteristic soft feel when applied on the skin. Emulsion lotions are stabilized using polymeric thickeners such as carbomers since the thermal stability of emulsion lotions is inferior to that of creams due to a lower viscosity. But these polymers do not generally make small emulsion droplets. Basically emulsion lotions have very similar compositions to that of emulsion creams except for having a much smaller amount of wax with a high melting point and oil soluble components. Therefore, emulsion lotions have very similar effects to those of cream.

According to the compositions in Table 9, oil soluble components, raw materials 1-11, were poured into a manufacturing tank and melted on heating to 70° C. Subsequently, emulsifiers, raw materials 12-14, were added and dissolved in the tank and was maintained at 50° C. Next, water soluble components, raw materials 17-26, dissolved previously in a supplementary tank at 50° C. were added to the manufacturing tank. Then, the high viscosity 2-phase complex of oil soluble phase and water soluble phase was formed by slowly mixing with a propeller mixer (1,000-8,000 RPM) upon increasing the temperature to the O-point. Homogeneous emulsion compositions were prepared by high speed mixing of the previous high viscosity complex with a propeller mixer at 1 atmospheric pressure, and then cooling to 50° C. Next, raw material 27 was added to the homogeneous emulsion composition, which was stirred again at 1,000-8,000 RPM. Finally, o/w nanoemulsion cosmetic moisture lotion was prepared by stirring the homogeneous emulsion composition homogeneously after adding oil soluble components, raw materials 15-16 and cooling to 30° C. Compositions of Comparative Examples 8-9 were prepared by the same method employed in those of Examples 8-9.

TABLE 9

| | | | | Comparative | |
|---|---|---|---|---|---|
| | | Example | | Example | |
| Component | | 8 | 9 | 8 | 9 |
| 1 | Microcrystalline wax | 1.0 | 1.0 | 1.0 | 1.0 |
| 2 | Vaseline | 0.5 | 0.5 | 0.5 | 0.5 |
| 3 | Bees wax | 0.5 | 0.5 | 0.5 | 0.5 |
| 4 | Cetostearyl alcohol | 2.0 | 2.0 | 2.0 | 2.0 |
| 5 | Glyceryl monostearate | 1.2 | 1.2 | 1.2 | 1.2 |
| 6 | Liquid paraffin | 22.0 | 22.0 | 22.0 | 22.0 |
| 7 | Neobee M-5 | 8.0 | 8.0 | 8.0 | 8.0 |
| 8 | Eutanol G | 5.0 | 5.0 | 5.0 | 5.0 |
| 9 | Shear butter | 0.5 | 0.5 | 0.5 | 0.5 |
| 10 | Preservative | Q.S. | Q.S. | Q.S. | Q.S. |
| 11 | Antioxidant | Q.S. | Q.S. | Q.S. | Q.S. |
| 12 | POP(20)-POE(50) vitamin E | 2.5 | 2.0 | 2.5 | 2.5 |
| 13 | POP(10)-POE(30) vitamin E | 0.5 | 0.5 | 0.5 | 0.5 |
| 14 | POP(100)-POE(20) vitamin E | 0.3 | 0.3 | 0.3 | 0.3 |
| 15 | Vitamin E Acetate | 0.3 | 0.3 | 0.3 | 0.3 |
| 16 | Perfume | Q.S. | Q.S. | Q.S. | Q.S. |
| 17 | Butylene glycol | 6.0 | 6.0 | 6.0 | 6.0 |
| 18 | Allantoin | 0.1 | 0.1 | 0.1 | 0.1 |
| 19 | Hyaluronic acid | 1.0 | 1.0 | 1.0 | 1.0 |
| 20 | Betaglucan | 0.1 | 0.1 | 0.1 | 0.1 |
| 21 | Rosemary extract | 5.0 | 5.0 | 5.0 | 5.0 |
| 22 | Cnidium officinale extract | 5.0 | 5.0 | 5.0 | 5.0 |
| 23 | Carbopol 934 | 0.1 | — | — | — |
| | Pemulen TR-2 | — | 0.1 | — | — |
| 24 | Natrosol 250 HR | — | — | 0.1 | — |
| 25 | Triethanolamine | 0.1 | 0.1 | — | — |
| 26 | Deionized water-1 | 25 | 25 | 25 | 25 |
| 27 | Deionized water-2 | to 100 | to 100 | to 100 | to 100 |
| | ?₀₁ (Weight)/?₀₂ (Weight) | 0.62/ | 0.62/ | 0.62/ | 0.62/ |
| | [Oil soluble phase/ | 0.42 | 0.42 | 0.42 | 0.42 |
| | (Oil soluble phase + Water | | | | |
| | soluble phase)] | | | | |
| | Θ₁-Point, ° C. | 71 | 72 | 70 | 71 |

### Experimental Examples 8-9

### Evaluation of Nanoemulsion Stability by Measuring Droplet Sizes and the Changes of Droplet Sizes

For confirming droplet sizes and evaluating the stability of o/w nanoemulsion cosmetic moisture lotion compositions, the droplet sizes of compositions in Examples 8-9 and Compositions Examples 8-9 were measured by the same method employed in Experimental Example 1 in Examples 1-4. The emulsion droplet diameter test results of o/w nanoemulsion moisture lotion compositions in Examples 8-9 and those in Comparative Examples 8-9 in Table 9, 1 day after preparation and 6 months after storing at 40° C. are shown in Table 10.

TABLE 10

Droplet size, nm

| | 1 day | 6 months | |
|---|---|---|---|
| Formulation | after storing | after storing | Droplet size |
| No. | preparation | at 40° C. | increase (%) |
| Example 8 | 85 | 88 | 3.5 |
| Example 9 | 87 | 92 | 5.7 |
| Comparative | 316 | 336 | 8.2 |
| Example 8 | | | |
| Comparative | 350 | 382 | 9.1 |
| Example 9 | | | |

As shown in Table 10, the mean droplet diameters of nanoemulsion compositions in Examples 8-9 prepared by simple mixing using a propeller stirrer 1 day after and 6 month after storing at 40° C. in this experiment were below 100 nm and the increase (%) in emulsion droplet sizes 6 months after storing at 40° C. had a relatively low value of 4.6%. On the other hand, the mean droplet diameter of nanoemulsion compositions prepared in Comparative Examples 8-9 was 333 nm, which is also in the droplet size range of nanoemulsions but much larger than that of compositions in Examples 8-9.

### Examples 10-11 and Comparative Examples 10-11

### O/W Nanoemulsion Skin Depigmentation Cream Compositions

According to compositions in Table 11, oil soluble components, raw materials 1-9, were poured into a manufacturing tank and melted on heating to 70° C. Subsequently, emulsifiers, raw materials 10-11, were added and dissolved in the tank and the tank was maintained at 50° C. Next, water soluble components, raw materials 12-19 and 22, dissolved previously in a supplementary tank at 50° C. was added to the manufacturing tank.

Then, the 2 phase complex of high viscosity of oil soluble phase and water soluble phase was formed by slowly mixing using a disperser. Next, homogeneous emulsion compositions were prepared by high speed mixing of the previous complex using a disperser (1,000-8,000 RPM) at 1 atmospheric pressure and cooling to 50° C. Next, raw material 20 previously dissolved in deionized water was added to the homogeneous emulsion composition. Finally, o/w nanoemulsion cosmetic moisture lotion was prepared by stirring the above emulsion composition homogeneously using a disperser followed by adding raw material 21 to adjust the pH to 4.0 to 6.0 and cooling to 30° C. Compositions in Comparative Examples 10-11 were prepared by the same method employed in those in Examples 10-11.

TABLE 11

| | | | | Comparative | |
|---|---|---|---|---|---|
| | | Example | | Example | |
| Component | | 10 | 11 | 10 | 11 |
| 1 | Bees wax | 4.0 | 4.0 | 4.0 | 4.0 |
| 2 | Paraffin was | 3.0 | 3.0 | 3.0 | 3.0 |
| 3 | Cetostearyl alcohol | 2.0 | 2.0 | 2.0 | 2.0 |
| 4 | Glyceryl monostearate | 1.0 | 1.0 | 1.0 | 1.0 |
| 5 | Liquid paraffin | 25.0 | 25.0 | 25.0 | 25.0 |
| 6 | Squalane | 20.0 | 20.0 | 20.0 | 20.0 |
| 7 | Dimethylpolysiloxane | 0.5 | 0.5 | 0.5 | 0.5 |
| 8 | Preservative | Q.S. | Q.S. | Q.S. | Q.S. |
| 9 | Antioxidant | Q.S. | Q.S. | Q.S. | Q.S. |
| 10 | POP(25)-POE(60) vitamin E | 2.5 | 3.0 | 2.5 | 3.0 |
| 11 | POP(5)-POE(0) vitamin E | 0.5 | 0.5 | 0.5 | 0.5 |
| 12 | Carbopol 934 | 0.4 | 0.06 | — | — |
| 13 | Veegum HV | — | 0.2 | — | 0.2 |
| 14 | Sodium metabisulfite | 0.2 | 0.2 | 0.2 | 0.2 |
| 15 | Sorbic acid | 0.2 | 0.2 | 0.2 | 0.2 |
| 16 | Glycerin | 7.0 | 7.0 | 7.0 | 7.0 |
| 17 | Propylene glycol | 5.0 | 5.0 | 5.0 | 5.0 |
| 18 | Triethanolamine | 0.1 | — | — | — |
| 19 | EDTA-2Na | 0.1 | 0.1 | 0.1 | 0.1 |
| 20 | Hydroquinone | 4.0 | 4.0 | 4.0 | 4.0 |
| 21 | Citric acid | Q.S. | Q.S. | Q.S. | Q.S. |
| 22 | Deionized wate | to 100 | to 100 | to 100 | to 100 |

| | | | | |
|---|---|---|---|---|
| Φ₀(Weight) [Oil soluble phase/ | 0.57 | 0.58 | 0.57 | 0.58 |
| (Oil soluble phase + Water | | | | |
| soluble phase)] | | | | |
| Θ-Point, ° C. | 72 | 73 | 72 | 76 |

### Experimental Example 1

### Evaluation of Nanoemulsion Stability by Measuring Emulsion Droplet Sizes and the Changes of Droplet Sizes

For confirming droplet sizes and evaluating the stability of o/w nanoemulsion skin depigmentation cream compositions in Examples 10-11 the droplet sizes of compositions in Examples 10-11 and those in Comparative Examples 10-11 were measured by the same method employed in Experimental Example 1 of Examples 1-4.

In Table 12, the test results of droplet sizes of compositions in Examples 10-11 and Comparative Examples 10-11 in Table 11 are shown.

TABLE 12

Droplet size, nm

| | 1 day | 6 months | |
|---|---|---|---|
| Formulation | after storing | after storing | Droplet size |
| No. | preparation | at 40° C. | increase (%) |
| Example 10 | 65 | 68 | 4.6 |
| Example 11 | 72 | 71 | 0.0 |
| Comparative | 313 | 335 | 7.0 |
| Example 10 | | | |
| Comparative | 257 | 278 | 8.1 |
| Example 11 | | | |

As shown in Table 12, the droplet diameter of o/w nanoemulsion cosmetic skin depigmentation cream compositions in Examples 10-11 is 69 nm, which is a very close value to the minimum diameter of nanoemulsions, 50 nm.

On the other hand, the mean droplet diameter of compositions in Comparative Examples 10-11 is 307 nm, which is much larger than that of compositions in Examples 10-11. And the mean droplet size increase (%) of nanoemulsions in Examples 10-11 is 2.3% and that of Comparative Examples 10-11 is 7.0%, respectively.

### Examples 12-13 and Comparative Examples 12-13

### O/w Nanoemulsion Acne Skin Treating Cream Compositions

According to the compositions in Table 13, oil soluble raw materials 1-9 and emulsifiers, raw materials 10-14, were poured into a manufacturing tank, which was heated to 70° C. Then, a high viscosity complex of an oil soluble phase and a water soluble phase was prepared by slowly mixing using a homogenizer after adding the water soluble raw materials 17-22 previously dispersed homogeneously in a complementary tank. Next, o/w nanoemulsion acne skin treating cream was prepared by stirring the mixture of the previous homogeneous complex using a propeller mixer after adding raw materials 15-16 and cooling to 30° C.

TABLE 13

| | | | | Comparative | |
|---|---|---|---|---|---|
| | | Example | | Example | |
| Component | | 12 | 13 | 12 | 13 |
| 1 | Paraffin | 3.0 | 3.0 | 3.0 | 3.0 |
| 2 | Bees wax | 3.5 | 3.5 | 3.5 | 3.5 |
| 3 | Shear butter | 1.0 | 1.0 | 1.0 | 1.0 |
| 4 | Glyceryl monostearate | 1.3 | 1.3 | 1.3 | 1.3 |
| 5 | Liquid paraffin | 25.0 | 25.0 | 25.0 | 25.0 |
| 6 | Squalane | 15.0 | 15.0 | 15.0 | 15.0 |
| 7 | Eutanol G | 5.0 | 5.0 | 5.0 | 5.0 |
| 8 | Phenoxythanol | 0.2 | 0.2 | 0.2 | 0.2 |
| 9 | Polydimethylsiloxane | 0.5 | 0.5 | 0.5 | 0.5 |
| 10 | POP(20)-POE(50) vitamin E | 2.5 | 3.5 | 2.5 | 3.5 |
| 11 | POP(100)-POE(200) | 0.5 | 0.5 | 0.5 | 0.5 |
| | vitamin E | | | | |
| 12 | Perfume | Q.S. | Q.S. | Q.S. | Q.S. |
| 13 | Sorbic acid | 0.2 | 0.2 | 0.2 | 0.2 |
| 14 | Sulfur | 0.5 | 0.5 | 0.5 | 0.5 |
| 15 | Acritamer 941 | 0.1 | 0.15 | — | — |
| 16 | Triethanolamine | 0.1 | — | — | — |
| 17 | Natrosol 250HR | — | — | 0.2 | 0.2 |
| 18 | Butylene glycol | 7.0 | 7.0 | 7.0 | 7.0 |
| 19 | Zinc oxide | 2.0 | 2.0 | 2.0 | 2.0 |
| 20 | Sodium metasulfate | 0.1 | 0.1 | 0.1 | 0.1 |
| 21 | Lactic acid | 0.5 | 0.5 | 0.5 | 0.5 |
| 22 | Deionized water | to 100 | to 100 | to 100 | to 100 |

| | | | | |
|---|---|---|---|---|
| Φ₀(Weight) [Oil soluble phase/ | 0.56 | 0.67 | 0.56 | 0.57 |
| (Oil soluble phase + Water | | | | |
| soluble phase)] | | | | |
| Θ-Point, ° C. | 75 | 72 | 76 | 75 |

### Experimental Example 1

### Evaluation of Nanoemulsion Stability by Measuring Emulsion Droplet Sizes and the Changes of Droplet Sizes

For confirming droplet sizes and evaluating the stability of o/w nanoemulsion acne skin treating cream compositions, droplet sizes of compositions in Examples 12-13 and those in Comparative Examples 12-13 were measured by the same method employed in Experimental Example 1 of Example 1-4.

The test results of droplet size measurement of o/w nanoemulsion cosmetic acne skin treating cream compositions 1 day after preparation and 6 months after storing at 40° C. are shown in Table 14.

TABLE 14

Droplet size, nm

| | 1 day | 6 months | |
|---|---|---|---|
| Formulation | after storing | after storing | Droplet size |
| No. | preparation | at 40° C. | increase (%) |
| Example 12 | 95 | 102 | 7.4 |
| Example 13 | 89 | 96 | 6.3 |
| Comparative | 427 | 486 | 13.8 |
| Example 12 | | | |
| Comparative | 459 | 503 | 9.5 |
| Example 13 | | | |

As shown in Table 14 the mean droplet diameter of o/w nanoemulsion acne skin treating cream compositions in Example 12-13 is 92 nm, which is close to the minimum value of nanoemulsion droplet size range of 20 to 50 nm for embodiments of the invention. On the other hand the mean droplet diameter for Comparative Examples 12-13 is 443 nm, which is close to the maximum value of nanoemulsion droplet size, 500 nm.

The mean droplet size increase (%) of nanoemulsions in Examples 12-13, 6 months after storing at 40° C. is 6.9% but that of Comparative Examples 12-13, 6 months after storing at 40° C. is 11.7%.

In the foregoing discussion, the term “Comparative Examples” do not implicate that these are prior art and therefore do not constitute an admission of prior art.

According to embodiments of the invention, nanoemulsions containing POP-POE vitamin E as emulsifiers and polyacrylic acid or polyacrylic acid derivative crosspolymers as emulsion assistants have a very small mean droplet diameter ranging from 43 to 96 nm and are very stable and safe. By the nanoemulsions described above, nanoemulsions with a much smaller mean droplet diameter have been obtained than for nanoemulsions prepared without emulsion assistants, polyacrylic acid or polyacrylic acid derivative crosspolymers by emulsifying using a simple high speed mixer such as a propeller, a disperser or a homogenizer due to the much improved properties when forming viscoelastic complex of oil soluble phase and water soluble phase above 40° C.

The economical productivity of embodiments of the invention is very high since the nanoemulsion cosmetic compositions could be prepared by simple mixing at 1 atmospheric pressure for embodiments of the invention. The nanoemulsions described above are not limited to a particular use but are applicable to several industrial areas such as cosmetics, foods, topical treatments, etc. The nanoemulsion cosmetic compositions according to embodiments of the invention also have good effects such as skin absorption, skin softening, skin moisturizing, skin firming, improvements in itchy or dry skin and good spreadability when applied on the skin. Particularly, nanoemulsion compositions according to embodiments of the invention have the advantage of providing a long lasting effect of skin treatment due to the favorable penetration of active drug components into the skin.

What is claimed is:

1. A nanoemulsion comprising:
a crosspolymer comprising at least one of a polyacrylic acid chain and a derivative of the polyacrylic acid chain; and
a compound represented by Formula 1: 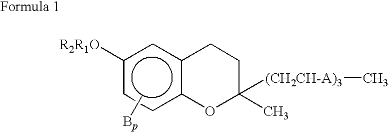
wherein R₁ is —(O—CH₂—CH₂)ₙ—, m is an integer from 0 to 300,
wherein R₂ is H—(O—CH(CH₃)—CH₂)ₙ—, n is an integer from 1 to 250,
wherein A is —CH₂—CH(CH₃)— or —C(CH3)=CH—, and
wherein B is —CH3, and p is 1, 2 or 3.

2. The nanoemulsion according to claim 1, wherein the crosspolymer comprises at least one selected from the group consisting of: a crosspolymer of a polyacrylic acid chain of Formula 2 crosslinked with an allyl ether of a polylol; a crosspolymer of a polyacrylic acid chain of Formula 2 crosslinked with an allyl ether of a propylene, and a C₆-C₄₀ alkyl acrylate polymer: 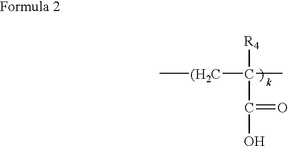
wherein R₄ is H or CH₃, and k is an integer from about 10 to about 100,000.

3. The nanoemulsion according to claim 2, wherein the polyol is a pentaerythritol or a sucrose.

4. The nanoemulsion according to claim 1, wherein the compound of Formula 1 comprises a natural vitamin E or a synthetic vitamin E.

5. The nanoemulsion according to claim 1, wherein the compound of Formula 1 is present in an amount from about 0.5 to about 60 wt. % with reference to the total weight of the nanoemulsion.

6. The nanoemulsion according to claim 1, wherein the compound of Formula 1 is present in an amount from about 1 to about 30 wt. % with reference to the total weight of the nanoemulsion.

7. The nanoemulsion according to claim 1, wherein the crosspolymer is present in an amount from about 0.01 to about 40% with reference to the total weight of the nanoemulsion.

8. The nanoemulsion according to claim 1, wherein the crosspolymer is present in an amount from about 0.02 to about 20% with reference to the total weight of the nanoemulsion.

9. The nanoemulsion according to claim 1, wherein the nanoemulsion comprises a droplet with a diameter in the range of about 43 to about 96 nm

10. The nanoemulsion according to claim 1, wherein at least a part of the droplet has a diameter of about 43 nm.

11. A cosmetic composition comprising the nanoemulsion according to claim 1.

12. The cosmetic composition according to claim 11, wherein the cosmetic composition is in the form of a cream or a lotion for applying to skin.

13. The nanoemulsion according to claim 1 further comprising at least one hydrophobic material selected from the group consisting of: paraffin oil, squalane, caprylic triglyceride, capric triglyceride, cetyloctanoate, octyldodecanol, isopropyl palmitate, jojoba oil, olive oil, safflower oil, evening primrose oil, Chinese pepper oil, sesame oil, shark oil, and oil soluble vitamins.

14. The nanoemulsion according to claim 1 further comprising at least one hydrophilic material selected from the group consisting of: propylene glycol, butylene glycol, glycerine, polyethylene glycol, hyaluronic acid, condroitin sulfate, glucosamine, vitamin C and panthenol.

15. A method of treating skin, comprising:
providing the nanoemulsion of claim 1; and
applying the nanoemulsion to skin.

16. A method of preparing the nanoemulsion according to claim 1, the method comprising:
providing a hydrophobic material, a hydrophilic material, the crosspolymer and the compound represented by Formula 1; and
mixing the hydrophobic material, the hydrophilic material, the crosspolymer and the compound represented by Formula 1.

17. The method according to claim 16, wherein the weight ratio (φ) of the hydrophobic material to the total weight of the hydrophobic and hydrophilic materials is from about 0.4 to about 0.75.

18. The method according to claim 17, wherein the high speed mixing occurs at 1 atmospheric pressure, and is not a high-pressure homogenizer.

19. A nanoemulsion comprising a compound represented by Formula 1: 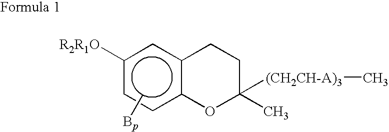
wherein R₁ is —(O—CH₂—CH₂)ₘ—, m is an integer from 0 to 300,
wherein R₂ is H—(O—CH(CH₃)—CH₂)ₙ—, n is an integer from 1 to 250,
wherein A is —CH₂—CH(CH₃)— or —C(CH3)=CH—,
wherein B is —CH3, and p is 1, 2 or 3, and
wherein the nanoemulsion forms a droplet with a diameter in the range of about 43 nm to about 96 nm.

20. A cosmetic composition comprising the nanoemulsion according to claim 20.

21. A method of treating skin, comprising:
providing the nanoemulsion of claim 20; and
applying the nanoemulsion to skin.

22. A nanoemulsion comprising a compound represented by Formula 1: 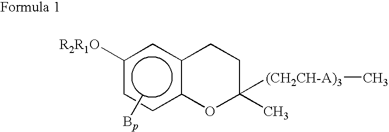
wherein R₁ is —(O—CH₂—CH₂)ₘ—, m is an integer from 0 to 300,
wherein R₂ is H—(O—CH(CH₃)—CH₂)ₙ—, n is an integer from 1 to 250,
wherein A is —CH₂—CH(CH₃)— or —C(CH3)=CH—, and
wherein B is —CH₃, and p is 1, 2 or 3.

23. A cosmetic composition comprising the nanoemulsion according to claim 23.

24. A method of treating skin, comprising:
providing the nanoemulsion of claim 23; and
applying the nanoemulsion to skin.
